(19) United States
(12) Patent Application Publication
(10) Pub. No.: US 2011/0144024 A1
(43) Pub. Date: Jun. 16, 2011

(54) PREVENTION AND TREATMENT OF RADIATION INJURY

(75) Inventors: Karl-Uwe Petersen, Aachen (DE); Martin Hauer-Jensen, Little Rock, AR (US)

(73) Assignee: PAION Deutschland GmbH, Aachen (DE)

(21) Appl. No.: 13/054,756

(22) PCT Filed: Sep. 23, 2008
(86) PCT No.: PCT/EP08/08043

§ 371 Date: Feb. 22, 2011

Related U.S. Application Data
(60) Provisional application No. 61/081,081, filed on Jul. 16, 2008.

(51) Int. Cl. A61K 38/36; C07K 14/745; A61P 39/00

(52) U.S. Cl. 514/13.7; 530/381

(57) ABSTRACT

The invention relates to the field of radiation injury. More particularly, this invention relates to the protection against/prevention of and treatment of diseases caused by ionizing radiation by the use of thrombomodulin.

The invention relates to the field of radiation injury. More particularly, this invention relates to the protection against/prevention of and treatment of diseases caused by ionizing radiation.

Ionizing radiation consists of highly-energetic particles or waves that can detach (ionize) at least one electron from an atom or molecule. Ionizing ability depends on the energy of individual particles or waves, and not on their number.

Ionizing radiation is produced by radioactive decay, nuclear fission and nuclear fusion and by particle accelerator that may produce, for example, fast electrons or protons or synchrotron radiation. An excessive exposure to ionizing radiation causes damage to organ tissue which is defined as “radiation poisoning” or “radiation sickness”. The clinical picture is summarized as “acute radiation syndrom”. A prolonged high level of exposure can also lead to a chronic radiation syndrome.

Typical symptoms of excessive exposure of a mammal (e.g. human) to ionizing radiation includes, within a few hours after exposure, a transient and inconsistent erythema (associated with itching). Then, a latent phase may follow, which lasts from a few days up to several weeks, when intense reddening, blistering, and ulceration or necrosis of the exposed tissue may occur. Very large skin doses can cause permanent hair loss, damaged sebaceous and sweat glands, atrophy, fibrosis, decreased or increased skin pigmentation, and ulceration or necrosis of the exposed tissue.

Situations that lead to excessive exposure to ionizing radiation include radioactive contamination by nuclear weapons or radioactive fall out, typically however radiation therapy, in particular in the course of cancer treatment.

There are currently more than 10 million cancer survivors in the United States. The exponential increase in the number of cancer survivors has led to a stronger focus on reducing treatment-related side effects, thus prompting a more proactive approach aimed at acquiring a better understanding of the molecular and cellular basis of treatment-related side effects, and at developing interventions to ameliorate or prevent long term toxicities of cancer therapy.

Approximately 70% of all cancer patients receive radiation therapy at some point during the course of treatment; and radiation injury thus plays a critical role in 25% of all cancer cures (De Vita et al., “Cancer: Principles and Practice of Oncology.” Philadelphia: Lippincott Williams & Wilkins, 2005). Recent advances in treatment delivery, such as the development of dose sculpting techniques, have led to an overall reduction in healthy tissue exposure during radiation therapy. Nevertheless, healthy tissue radiation toxicity so far remains the most important dose limiting factor in radiation injury and a major obstacle to radiotherapy.

Severe side effect are associated with current radiation-therapy, including myelosuppresion, acute and chronic radiodermitis, mucositis, xerostomia, enteritis, acute and chronic proctitis, hepatitis, pneumopathy, pericarditis, cardiomyopathy, acute glomerolunephritis and chronic glomerulosclerosis.

Presently there is only one radioprotective drug available; namely the radical scavenger Amifostine (Medimmune). Other treatments focus on symptomatic curing only.

Thus, despite long-standing need for the development of methods to prevent or treat tissue radiation toxicity, progress has been slow, and methods that are safe and effective are still not available (Hauer-Jensen et al: “Bowel injury: current and evolving management strategies.” Semin Radiat Oncol 2003; 13: 189-202).

Consequently there remains a great need for a radioprotective agent that can be made available either before or after radiation injury, or both.

Thus, it is an objective of the present invention to provide novel means for the treatment of radiation injury.

This objective is solved by providing a medicament for treating a patient suffering from or believed to be suffering from radiation injury or being subject of ongoing or future radiation therapy or any other kind of exposure to ionizing radiation comprising a therapeutically effective amount of thrombomodulin or thrombomodulin analogue, in particular an oxidation resistant thrombomodulin analogue.

As used herein the term “radiation injury” shall refer to an injury or damage that is caused by exposure to ionizing radiation. Radiation injury includes but is not limited to radiation poisoning, radiation sickness, acute radiation syndrome or chronic radiation syndrome.

As used herein, the term “ionizing radiation” refers to radiation that has sufficient energy to eject one or more orbital electrons from an atome or molecule (e.g. a particles, β particles, γ rays, x-rays, neutrons, protons and other particles having sufficient energy to produce ion pairs in matter).

A “therapeutically effective amount” is defined as the amount of active ingredient that will reduce the symptoms associated with radiation injury or prevent or mitigate symptoms of radiation injury after subsequent exposure to ionizing radiation. “Therapeutically effective” also refers to any improvement in disorder occurrence, severity, frequency or duration of incidences compared to no treatment. The term “treatment” encompasses either curing or healing as well as mitigation, remission or prevention, unless otherwise explicitly mentioned.

In the natural environment thrombomodulin is a membrane protein that acts as a thrombin receptor on the endothelial cells lining the blood vessels. Thrombin is a central enzyme in the coagulation cascade, which converts fibrinogen to fibrin, the matrix clots are made of. Initially, a local injury leads to the generation of small amounts of thrombin from its inactive precursor, prothrombin. Thrombin, in turn, activates platelets and, second, certain coagulation factors including factors V and VIII. The latter action gives rise to the so-called thrombin burst, a massive activation of additional thrombin molecules, which finally results in the formation of a stable clot.

When bound to thrombomodulin, however, the activity of thrombin is changed as follows: A major feature of the thrombin-thrombomodulin complex is its ability to activate protein C, which then downregulates the coagulation cascade by proteolytically inactivating the essential cofactors Factor Va and Factor VIIIa (Esmon et al., Ann. N.Y. Acad. Sci. (1991), 614:30-43), thus affording anticoagulant activity. The thrombin-thrombomodulin complex is also able to activate the thrombin-activatable fibrinolysis inhibitor (TAFI), which then antagonizes fibrinolysis. Furthermore, activated protein C is known to exhibit anti-inflammatory properties.

Mature TM is composed of five domains: an N-terminal lectin-like binding domain, an epidermal growth factor (EGF) domain which consists of 6 EGF-like-repeats, a Ser/Thr-rich region (O-linked glycosylation domain), a transmembrane domain and a cytoplasmic domain.

A domain is a three dimensional, self-assembling array of amino acids of a protein molecule, which contains structural elements necessary for a specific biological activity of that protein. While the thrombin binding property is thought to be linked to the EGF domain, the lectin-like domain is believed to be responsible for the direct anti-inflammatory properties of TM (van de Wouwer et. al: The lectin-like domain of thrombomodulin interferes with complement activation and protects against arthritis; in: Journal of Thrombosis and Haemostasis 4, 1813-1824, 2006).

| approximate amino | |
|---|---|
| acid position | Domain |
| −18-1 | signal peptide |
| 1-226 | N-terminal domain - homologous to some |
| | lectins (lectin-like domain) |
| 227-462 | repeats of EGF-like domains (EGF domain) |
| 463-497 | O-linked glycosylation domain |
| 498-521 | stop transfer domain - membrane spanning |
| 522-557 | Cytoplasmic domain |

(see C. S. Yost, et al, (1983) Cell 34:759-766 and D. Wen, et al, (1987) Biochemistry 26:4350-4357, both herein incorporated by reference.)

The term “native” thrombomodulin refers to both the natural protein and its soluble form having substantially the same characteristic biological activities of membrane-bound or detergent solubilized (natural) thrombomodulin. These soluble peptides are also referred to as “wild-type” or “non-mutant” peptides. The biological activity includes the ability to act as a receptor for thrombin, and therewith increase the activation of protein C, and other biological activities associated with native thrombomodulin, in particular anti-inflammatory activities such as the suppression of the complement system. The assessment of the antthrombotic properties of TM analogues is known to the skilled person. Assessing the suppression of the complement system is disclosed e.g. in the above publication of van de Wouwer et al.

The term “thrombomodulin analogue” in contrast refers to proteins which differ from the native TM by at least one amino acid, either by deletion, substitution, derivatisation or addition thereof, or any other kind of modification, whereas their activity substantially corresponds to the biological activities of native TM in terms of activation of protein C or activation of anti-inflammatory processes, in particular the suppression of the complement system. In one embodiment the TM analogues of the invention exhibit substantially the same properties as the native TM in both terms, i.e. regarding the activation of protein C and the induction of anti-inflammatory processes.

TM analogues applicable according to the invention can comprise one or more domains of the native TM. In one embodiment TM analogues can be used which are composed by the EGF and the lectin-like domain only. However, a TM analogue consisting of the EGF domain only can also be used. Such TM analogues are disclosed in WO 2008/073884 A1.

As outline above, in one embodiment of the invention the TM analogue has the amino acid amino sequence of the native sequence or parts thereof with one or more amino acids removed or replaced by one or more different amino acids. Specifically, the amino acids removed or replaced are either one or both of the methionine residues at positions 291 or 388 in the native TM protein sequence (SEQ ID No1). These methionines can be replaced with the amino acid alanine, leucine, isoleucine, glutamine or valin. Most preferred is the substitution with leucine.

Replacing these methionines results in an oxidation resistant TM analogue that retains its activity after exposure to oxidants, in particular the activation of protein C. Its anti-inflammatory activities are not affected. It may also exhibit an increased specific activity when compared to an equivalent peptide not having an amino acid substitution. “Oxidation resistant TM analogue” thus are peptides which are resistant against inactivation by oxidants, such as oxygen radicals. Preferably these TM analogues are soluble.

Hence in one embodiment of the invention a thrombomodulin analogue can be used that has the characteristic antithrombotic and anti-inflammatory activity of native thrombomodulin but which is soluble in aqueous solution and is not inactivated after having been exposed to oxidants. According to this embodiment of the invention the peptides are lacking at least the membrane spanning and cytoplasmic domains of native thrombomodulin.

Various forms of soluble thrombomodulin or thrombomodulin analogues are known to the skilled person, e.g. the so called ART-123 developed by Asahi Corporation (Tokyo, Japan) or the recombinant soluble human thrombomodulin known as solulin, currently under development by PAION Deutschland GmbH, Aachen (Germany). The recombinant soluble thrombomodulin, i.e. a soluble thrombomodulin without any modification of the amino acid sequence of the native, is subject of the EP 0 312 598. Both, ART-123 and solulin can be used according to the invention.

Solulin is a soluble, as well as protease and oxidation-resistant analogue of human thrombomodulin and thus exhibits a long life in vivo. Solulin's main feature lies in its broad mechanism of action since it not exclusively inhibits thrombin. It also activates the natural protein C pathway, and therefore stops further generation of thrombin.

Solulin is inter alia subject of the European patent 0 641 215 B1, EP 0 544 826 B1 as well as EP 0 527 821 B1. Solulin contains modifications compared to the sequence of native human thrombomodulin (SEQ. ID NO. 1) at the following positions: G −3V, Removal of amino acids 1-3, M388L, R456G, H457Q, S474A and termination at P490. This numbering system is in accordance with the native thrombomodulin of SEQ. ID NO. 1. The sequence of solulin as the preferred embodiment of the invention is shown in SEQ. ID NO. 2.

However, notably, according to the invention also thrombomodulin analogues can be utilized, which comprise only one or more of the above mentioned properties, or of the properties outlined in the above mentioned European patent documents EP 0 544 826 B1, EP 0 641 215 B1 and EP 0 527 821 B1.

Other TM analogues are known, e.g. from WO 2008/073884 A2, which are modified as to have a reduced ability to activate protein C. In one embodiment of the invention also these analogues can be used to treat IBD.

Particularly preferred thrombomodulin analogues applicable according to the invention are those that have one or more of the following characteristics:
(i) oxidation resistance
(ii) protease resistance
(iii) homogeneous N- or C-termini
(iv) post-translational modification e.g., by glycosylation of at least some of the glycosylation sites of native thrombomodulin (SEQ ID NO: 1)
(v) linear double-reciprocal thrombin binding properties
(vi) solubility in aqueous solution in relatively low amounts of detergents and typically lack a transmembrane sequence
(vii) lack of glycosaminoglycan chain.

Most preferred is a molecule comprising all of these modifications, e.g. as shown by solulin. The manufacture of these analogues is disclosed in the above mentioned European patent documents.

In a further embodiment of the invention, the thrombomodulin analogue known from the WO 01/98352 A2 or U.S. Pat. No. 6,632,791 can be used. Also thrombomodulin derived by rabbits can be used. The 6EGF fragment of solulin is an example of a thrombomodulin fragment with essentially the same biological activity regarding the formation of a complex with thrombin with the ability to activate human protein C. This fragment essentially consists of the six epithermal growth factors domain of native thrombomodulin.

The effects underlying the present invention were found when assessing mortality of test animals after exposure to whole body X-ray irradiation. Mice were irradiated with increasing doses of X-rays which resulted in an increased mortality of the control animals. The mice treated with a soluble TM however showed a decreased mortality in comparison to the untreated control group. These data were further confirmed by experiments showing that ionizing radiation of TM resulted in an inactivation of TM antithrombotic activity. This can be explained by an oxidation of Met388 in the EGF domain of TM (see the Examples below), which is the linker between the EGF domains 4 and 5 and which is known to be critical for TM function (Clarke et al.: The short loop between epidermal growth factor-like domains 4 and 5 is critical for human function. Journal of Biological Chemistry 268, 6309-6315 (1993).

In further experiments the inventors showed, that the TM analogue solulin is not inactivated by ionizing radiation. Consequently an oxidation resistant TM can be used either to treat or to prevent radiation injury.

It has been known for a long time that—besides induction of apoptosis due to free radical-mediated DNA damage—injury of the microvasculature plays a central role in early and delayed radiation responses in many normal tissues. The high radiation sensitivity of the microvasculature is to a large extent attributable to the endothelial cells, since radiation induces a plethora of morphological and functional alterations in endothelial cells, including apoptosis, detachment from the basement membrane, and increased endothelial permeability, resulting in fibrin deposition in the interstitial space.

Furthermore it is known from clinical and pre-clinical studies that radiation therapy causes a striking reduction in endothelial TM levels and that chemical oxidation can lead to an inactivation of TM (e.g. Richter et al: Is the loss of endothelial thrombomodulin involved in the mechanism of chronicity in late radiation enteropathy?, Radiother Oncol 1997, 44: 65-71; Glaser et al.: Oxidation of a specific methionine in thrombomodulin by activated neutrophil products blocks cofactor activity: J. Clin. Invest 90, 2565-2573 (1992). Thus it was suggested that “the restitution of the thrombomouldin-protein C pathway is an appealing strategy by which to prevent or treat normal tissue toxicity associated with radiation treatment of cancer” (see for more details Hauer-Jensen et al: Radiation injury and the protein C pathway, Crit. Care Med 2004, 32 (Suppl. 5), S325-S330).

However, so far any evidence or experimental support is lacking as to the validity of this hypothesis in vivo. Notably due to the complexity of the in vivo situation any estimation or assumption for the clinical setting solely derived from in vitro or cell free data is precluded. Additionally, many other inhibitors of blood clotting have been tested in the past in attempts to ameliorate normal tissue radiation toxicity. But only negative or inconsistent results have been obtained. For example, the anticoagulant heparin administered at the time of irradiation even exacerbates radiation-induced tissue injury (Wang et al., “Modulation of intestinal response to ionizing radiation by anticoagulant and non-anticoagulant heparins.” Thromb Haemost 2005; 94: 1054-1059). Thus researchers remained sceptical as to the therapeutic success of a possible “restitution of the TM-protein C” pathway.

However, as demonstrated by the in vivo experiments presented below the inventors now indeed found that TM and TM analogues, in particular oxidation resistant TM analogues, can be used for the treatment and prevention of radiation injury.

According to the invention TM or TM analogues can be administered for the treatment, in particular also for the prevention of acute or chronic radiation injury. The “acute treatment” means the administration of TM or TM analogues during an ongoing radiation treatment, in particular for cancer, while a “chronic treatment” by convention is considered to occur approximately three months or more after the termination of the treatment. Thus, in a further embodiment, TM or TM analogues can be given to cancer survivors to treat chronic effects of radiation therapy they have undergone previously or to cancer patients as a treatment concomitant to radiation therapy.

Furthermore TM or TM analogues can be administered to a patient, who will be subject of subsequent radiation therapy or any other exposure to ionizing radiation. In this aspect of the invention TM or TM analogues are used for the prevention of radiation injury. Even if a complete prevention is not possible the administration of TM or TM analogues prior to the radiation onset will mitigate later radiation injury.

In a particular embodiment of the invention TM or TM analogues, e.g. an oxidation resistant TM analogue, is given to a patient who receives a therapeutically effective amount of a cytotoxic agent, which—due its mode of action—directly or indirectly leads to the production of oxidants, such as free oxygen radicals. Advantageously, the oxidation resistant TM analogue allows the combined administration with such “oxidising” anti-cancer agents, therewith enabling the reduction of radiation injury effects even in the presence of cytotoxic agents able to precipitate the production of oxidants.

A combined treatment according to the invention can be a concomitant or a sequential administration of TM or TM analogues with cytotoxic agents (anti-cancer agents) with direct or indirect oxidising potential. Hence, according to one embodiment of the invention, a pharmaceutical composition is provided containing a therapeutically effective amount of TM or TM analogue (e.g. oxidation resistant TM analogue) and a cytotoxic agent. Possible cytotoxic agents are doxorubicin or dactinomycin.

In one embodiment of the invention thrombomodulin and TM analogues can be used to treat patients with an “acute radiation syndrome” (ARS) (sometimes known as radiation toxicity or radiation sickness), which is defined on the basis of the following conditions:
The radiation must be large (i.e. greater than 0.7 Gy). Mild symptoms may be observed with doses as low as 0.3 Gy.
The dose usually must be external (i.e., the source of radiation is outside of the patient's body).
The radiation must be penetrating (i.e., able to reach the internal organs).
The entire body or a significant portion of it must have received the dose.
The dose must be delivered in a short time (usually a matter of minutes).

Fractionated doses are often used in radiation therapy. These large total doses are delivered in small daily amounts over a period of time. Fractionated doses are less prone to induce ARS than a single dose of the same magnitude.

In a further embodiment of the invention, thrombomodulin and TM analogues can be used to treat patients with a bone marrow syndrome (sometimes referred to as hematopoietic syndrome) whereby the full syndrome will usually occur with a dose greater than approximately 0.7 Gy although mild symptoms may occur as low as 0.3 Gy.

In another embodiment of the invention, thrombomodulin and TM analogues can be used to treat patients with a cardiovascular (CV)/central nervous system (CNS) syndrome, whereby the full syndrome will usually occur with a dose greater than approximately 50 Gy although mild symptoms may occur as low as 20 Gy. Death occurs within 3 days due to the collapse of the circulatory system as well as increased pressure in the confining cranial vault as the result of increased fluid content caused by edema, vasculitis, and meningitis.

In a further embodiment of the invention thrombomodulin and TM analogues can be used to treat patients with cutaneous radiation syndrome that comprises a complex pathological syndrome resulting from acute exposure of the skin to radiation. The damage of the basal layer results in inflammation, erythema and dry or moist desquamation.

In a preferred embodiment of the invention, thrombomodulin and TM analogues can be used to treat patients with a gastrointestinal syndrome, whereby the full syndrome will usually occur with a dose greater than approximately 10 Gy, although some symptoms may occur at a dose as low as 6 Gy. For this syndrome, survival is extremely unlikely. Destructive and irreparable changes in the GI tract and bone marrow usually cause infection, dehydration and electrolyte imbalance. Death usually occurs within 2 weeks.

In a further embodiment of the invention thrombomodulin can be used therapeutically in combination with chemotherapeutic agents including alkylating agents (e.g. cyclophosphamide), platinum-containing agents (e.g. cisplatin) or intercalating agents (e.g. doxorubicin).

The present invention furthermore can be applied in all settings where ionizing radiation has the potential to cause cell injury, e.g. as a protectant against solar radiation, or as a protectant usable for humans potentially exposed to ionizing radiation, such as workers in nuclear power plant facilities or radioactive waste dump sites or accidents or, finally, in the event of military or terroristic attacks.

Thrombomodulin or TM analogues, in particular Solulin, preferably are given non-orally by parenteral e.g. intravenous or subcutaneous application. An intravenous bolus application is possible. It can be administered in multiple doses. A multiple or chronic administration of thrombomodulin or the TM analogues is possible.

### EXAMPLES

### I. Whole Body Irradiation Model in Mice

A whole body irradiation model was used to test the ability of soluble thrombomodulin (solulin) to decrease the radiation-induced mortality. The drug was added 30 min or 24 hours after irradiation.

### 1. Material and Methods

The study was conducted in male mice, CD2F1 strain. Two groups of animals consisting of 8 animals were subjected to a whole body irradiation of 8.5 Gy and two groups of the same size received a dosis of 9 Gy. A Cs137 γ-ray source was used for the radiation experiments. 30 min or 24 hours after irradiation, the control groups received an injection of 100 μl vehicle (PBS pH 7.0 and 5% mannitol buffer) into the tail vein, whereas the solulin group received an injection of 100 μl solulin (4 mg/kg dissolved in vehicle buffer) into the tail vein. The mice were checked twice daily (8:30 and 5:00 p.m.) and the number of surviving mice was recorded.

### 2. Results

Irradiation with 8.5 Gy:

When assessing the effect of solulin administration 30 min after radiation, 25% of the mice of the control group died, whereas after treatment with solulin only approximately 10% of the mice died. There was no difference between the solulin and control groups when solulin was administered 24 hours after radiation.

Irradiation with 9Gy:

Larger solulin effects were observed at the higher radiation level of 9 Gy. When treated with solulin 30 min after radiation, only 25% of the mice died, whereas the control group showed a mortality of more than 60%. No effect of solulin was obtained when it was administered at an interval of 24 hours.

Solulin was generally well tolerated.

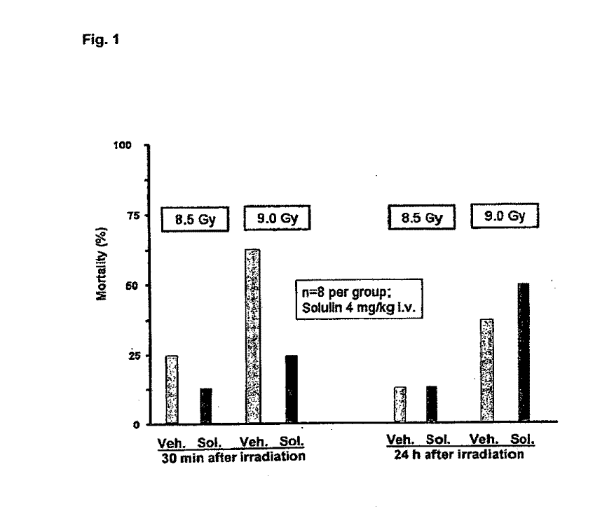

FIG. 1: Solulin decreases mortality after whole body irradiation in mice.

### II. In Vitro Irradiation of Solulin vs. Recombinant Human Thrombomodulin

Full length recombinant human thrombomodulin and solulin were exposed to γ irradiation in a cell-free system. The influence of radiation on functional activity was assessed with the protein C activation assay.

### 1. Material and Methods

Irradiation. Recombinant full length human TM was dissolved in buffer to a final concentration of 50 nM for the protein C activation assay. The buffer used for the assay contained 10 mM Tris-HCl, 0.2 M NaCl, 5 mM CaCl2, and 0.1% polyethylene glycol, at pH 8.0.

Samples were irradiated with a single or fractionated dose of 0, 10 or 20 Gy using a Gammacell 1000 Irradiator (Nordion International, Inc., Kanata, Ontario, Canada). The samples were exposed to a 137Cs source and received 5.90 Gy/min. For each dose 3 vials were used and the protein C activation assays were done in triplicates.

Protein C activation assay. Changes in TM functional activity were assessed by measuring protein C activation. Following irradiation, samples were diluted to a final concentration of 1.0 nmol/l TM and incubated with 200 nmol/l protein C and 1.4 nmol/l thrombin (180 minutes at 37° C., 1.2 ml total volume) in a 96-well plate to generate activated protein C. The amount of activated protein C generated was measured by monitoring hydrolysis of chromogenic substrate S-337 at 405 nm in a microplate reader (Bio-TEK Instruments, Winooski, Vt., USA) at 5-min intervals for the first 60 minutes and, where applicable, 30 minute intervals thereafter. The results were expressed as mean OD slope values (ΔOD/Δt) or as protein C generated at 60 minutes.

### 3. Results

The results clearly show that the activity of solulin is not reduced by radiation, neither in the single dose, nor in the fractionated dose.

### III. Inactivation of Thrombomodulin by Ionizing Radiation

Background: Normal tissue radiation injury is associated with loss of vascular thromboresistance, notably because of deficient levels of endothelial thrombomodulin (TM). TM is located on the luminal surface of most endothelial cells and serves critical anticoagulant and anti-inflammatory functions. Chemical oxidation of a specific methionine residue (Met388) at the thrombin-binding site in TM reduces its main functional activity, i.e., the ability to activate protein C. We examined whether exposure to ionizing radiation affects TM in a similar manner.

Methods: Full length recombinant human TM, a construct of epidermal growth factor-like domains 4-6 that are involved in protein C activation, and a synthetic peptide containing the methionine of interest, were exposed to gamma radiation in a cell-free system. ie, a system not confounded by TM turnover or ectodomain shedding. The influence of radiation on functional activity was assessed with the protein C activation assay; formation of TM-thrombin complex was assessed with surface plasmon resonance (Biacore), and oxidation of Met388 was assessed by HPLC and confirmed by mass spectroscopy.

Results: Exposure to radiation caused a dose dependent reduction in protein C activation, impaired TM-thrombin complex formation, and oxidation of Met388, all in a radiation dose dependent manner.

Conclusion: These data demonstrate that ionizing radiation adversely affects the TM molecule. Our findings may have relevance to normal tissue toxicity in clinical radiation therapy, as well as to the development of radiation syndromes in the non-therapeutic radiation exposure setting.

### Materials and Methods

All experiments were performed in a cell-free system. ie, in a system not confounded by transcriptional regulation or ectodomain shedding of TM. The functional activity of TM was assessed with the protein C activation assay, interaction between TM and thrombin was monitored using surface plasmon resonance (SPR), and oxidation of Met388 was assessed by high performance liquid chromatography (HPLC) with mass spectroscopy (MS) confirmation.

### Irradiation

All samples were dissolved in buffer (see below) and irradiated in 1 ml polypropylene microcentrifuge tubes (Cat # 02-681-374, Fisher Scientific, Pittsburgh, Pa.) in a total volume of 500 μL. The TM samples were stored at 4° C. as recommended by the supplier for optimal long term stability. Before irradiation, the samples were placed at room temperature for 1 hour to ensure stable and equal sample temperature. After irradiation, the samples were returned to 4° C. within 20 minutes, but no sooner than 15 minutes after irradiation. This protocol minimized intra- and inter-experimental variability and ensured that, the exposure to radiation (or sham-irradiation) was the only variable across all experiments.

Irradiation was performed in a Gammacell 1000, model B (with 2 cesium capsules) Cs-137 irradiator (Atomic Energy of Canada Ltd., Kanata, Ontario, Canada) with dose rate 5.90 Gy/minute. Constant placement of the samples within the 95% isodose area in the irradiator was ensured by placing the samples in a circular configuration on a round plastic rack with a capacity of 8 tubes, elevated by 25 mm, and centered on a turntable rotation at 4 rpm.

Samples were exposed to graded single or fractionated radiation doses, with the lowest radiation dose (1.77 Gy) being close to the fraction size of 1.8-2 Gy commonly used in clinical radiation therapy. At least 3 independent TM samples were irradiated at each dose level for all experiments, not including optimization and validation studies, and separate sets of samples were irradiated for each of the 3 endpoints (protein C activation assay, SPR, HPLC/MS).

### TM Activity

The effect of single dose irradiation on TM functional activity was assessed with the protein C activation assay after exposure to 0, 1.77 (0.3 min radiation exposure), 10 (1.7 min), 20 (3.4 min), 40 (6.8 min), and 80 Gy (13.6 min). The dose 1.77 Gy was used for practical reasons because the timer on the irradiator operated in 0.1 minute increments.

The studies were performed using a recombinant human TM molecule (Eli Lilly and Co., Indianapolis, Ind.) comprised of the extracellular portion of TM (the N-terminal lectin-binding domain, the 6 epidermal growth factor [EGF]-like repeats, and the serine/threonin-rich domain) without the transmembrane and intracellular domains and, importantly, without the chondroitin sulfate moiety. TM was dissolved to a final concentration of 50 nM in buffer containing 10 mM Tris-HCl, 0.2 M NaCl, 5 mMCaCl₂, and 0.1% polyethylene glycol, pH 8.0 (1).

The response to fractionated irradiation was investigated by comparing the activity of recombinant TM exposed to 6 or 12 fractions of 1.77 Gy with the activity of TM exposed to the same total radiation doses administered as single exposures (10.62 Gy [6×0.3 min] or 21.24 [12×0.3 min] Gy, respectively). Fractionated irradiation was performed with twice-daily fractions separated by 6 hours, and the experiment was designed to maintain the time between the last fraction and performance of the protein C assay constant for all samples. All samples were subjected to the same number of radiation/sham-irradiation procedures (i.e., each was irradiated or sham-irradiated 12 times). All samples for each experiment (fractionated as well as single dose irradiated) were transported to the irradiator together as one batch each time and irradiation was commenced after a uniform period of 1 hour. Samples to be sham-irradiated were treated in identical manner, placed in the irradiator's chamber for the same time as samples that were irradiated, but without activation of the source.

The radiation response of TM in the presence of myeloperoxidase (MPO) was also investigated. MPO is present in neutrophils and monocytes and is involved in the oxidative burst associated with inflammation, for example in the setting of post-radiation inflammation. MPO catalyzes the formation of hypochlorous acid, a potent oxidative agent, from H₂O₂ and Cl. TM samples, to which 1.0 μM MPO (Fischer Scientific, Suwanee, Ga.) had been added immediately before irradiation, were exposed to 0 Gy, 1.77 Gy, 20 Gy, or 80 Gy and subsequently analyzed by protein C assay to address the possibility of an additive or synergistic effect of MPO on radiation-induced Met388 oxidation, similar to what has been reported for chemical oxidation (15). The MPO concentration used in the present study was the same as that used by Glaser et al (1).

The protein C activation assay was performed as follows. Irradiated and sham-irradiated samples were diluted to a final concentration of 2.5 nmol/L TM and incubated with 200 nM protein C and 1.4 nM thrombin (60 minutes at 37° C., 1.2 mL total volume) in a 96-well plate to generate activated protein C. The amount of activated protein C generated was measured by monitoring hydrolysis of the chromogenic substrate, S-337, at 405 nm in a microplate reader (Bio-TEK Instruments, Winooski, Vt.) at 5-minute intervals for 60 minutes. The results were expressed as mean OD at 60 minutes. All assays were performed in triplicate and the average was considered a single value for statistical purposes.

### Thrombin-TM Complex Formation

The interaction between thrombin and thrombomodulin (TM) was monitored by SPR using a BIACore 3000 instrument (Biacore AB, Uppsala, Sweden). Recombinant full length human TM was dissolved to a final concentration of 50 nM or 100 nM in HBS buffer containing 20 mM HEPES, 150 mM NaCl, 2 mM CaCl₂ and 0.005% P20 (a surfactant) at pH 7.4. They were irradiated with 0 Gy, 1.77 Gy, 20 Gy, or 80 Gy as described above and subsequently analyzed using SPR.

In SPR, a light source is polarized and directed at a chip of gold film, the beam being reflected into an optical detection unit. The amount of mass bound to the chip surface changes the angle of reflection and, in turn, changes the refractive index close to the surface of the sensor chip. The detection unit detects the change in angle, referred to as the resonance angle. The change in resonance angle, in Biacore terms, is expressed in Resonance Units (RU), with 1000 RU corresponding to approximately 1 ng of bound protein per mm² chip surface. A sensogram is a continuous display of RU versus time in seconds, i.e., a real-time representation of mass associating and/or dissociating on the surface of the chip (16).

Thrombin was dissolved at 60 ng/μL in 10 mM sodium acetate buffer (pH 5.5) and immobilized on one channel of a CM-5 sensor chip (research grade, Biacore AB). Thrombin amine groups were covalently bound to activated carboxyl groups on the sensor chip using the Biacore Amine coupling kit according to the manufacturer's instructions and in accordance with the methods described by Kishida et al (2). Thrombin immobilization resulted in approximately 4000 resonance units (RU) bound to the chip surface. Another channel was activated and blocked with 1.0 M ethanolamine at pH 8.5 for six minutes at 5 μL/minute for use as a blank, without bound thrombin. Although the availability of free amine groups on lysines is important for thrombin-TM complex formation, the random nature of amine coupling, the many lysines in thrombin, and the high number of thrombin molecules bound to the chip ensured that a sufficient number of thrombin molecules would be available to bind TM.

Fifteen μL of 50 nM TM in HBS buffer was passed over the immobilized thrombin at a rate of 5 μL/minute. Sensograms were collected in real time as the difference between binding of the thrombin-containing channel and non-specific binding of the blank reference channel, i.e., RU values for the blank channel were subtracted from those of the thrombin-containing channel to account for non-specific binding of TM to the matrix. The association phase of the binding reaction consisted of a 3 minute injection of TM at a rate of 5 μL/minute, followed by a wash with HBS buffer for 4 minutes at 5 μL per minute to monitor the dissociation of TM. The chip surface was regenerated by washing with 1.5 M NaCl for 1 minute. The baseline was allowed to stabilize for 5 minutes before injection of the next sample. The change in response from baseline was measured for each sample and compared. After optimization of the SPR procedure, SPR was performed 3 times (on separate days) at each radiation dose level.

### Oxidation of Met388

To avoid confounding by methionines elsewhere in the TM molecule, a synthetic β-amino acid peptide containing Met388 (APIPHEPHRCQMF), and a TM fragment consisting of TM EGF-like domains four, five and six (TMEGF456). the region containing Met388 and responsible for protein C activation, were used to determine oxidation of Met388 by HPLC/MS.

Three separate samples were analyzed per radiation dose level. At each run, each sample was used for 3.4 injections, again with the average being considered a single value.

Peptide preparation. 5 mg of the peptide APIPHEPHRCQMF (synthesized by SIGMA Genosys) was dissolved in 500 μL of 1× phosphate buffered saline (PBS) pH 7.4 (1.46 mM KH₂PO₄, 9.9 mM Na₂HPO₄. 2.68 mM KCl, 0.137 M NaCl) then brought to a final concentration of 50 mM tris(2-carboxyethyl)-phosphine (TCEP) (Calbiochem, La Jolla, Calif.) by adding 25 μL of a 1 M stock of TCEP buffered with 750 mM potassium phosphate tribasic, to pH 7.0. Further purification was performed with HPLC under reducing conditions, while separating oxidized and un-oxidized peptide. The samples were placed in 1 mL borosilicate glass tubes (VWR International, West Chester, Pa.) until irradiated.

Purification of TMEGF456. A recombinant form of thrombomodulin epidermal growth factorlike domains four, five, and six (TMEGF456), ranging residues 365 to 481 and an additional N-terminal His-Met sequence, was expressed in P. pastoris as described by Wood et al (3). The expression system was the gracious gift of Dr. Elizabeth Komives at the University of California, San Diego. Upon completion of growth, cells were separated from the growth media by centrifugation and supernatants were decanted from the cell pellets and combined. After adding 1.8 g/L disodium EDTA (J. T. Baker, Phillipsburg, N.J.), the supernatant was pumped using a FMI Q model rotary piston pump (Fluid Metering, Inc., Syosset, N.Y.) at 5 mL/minute over a 10 mL bed volume of Q Sepharose FF (Amersham Biosciences, Piscataway, N.J.) packed into a 10×100 mm column, previously equilibrated with 4 bed volumes of 50 mM Tris-HCl, pH 7.1. Three elution buffers were prepared for a step gradient: Eluent A consisting of 50 mM Tris-HCl, pH 7.1, 0.625 M NaCl, eluent B. consisting of 50 mM Tris-HCl, pH 7.1, 1.25M NaCl, and eluent C consisting of 50 mM Tris-HCl, pH 7.1, 2M NaCl. The three buffers were pumped over the column in series, each for 10 minutes at 5 mL/minute, and collected separately. Eluent B, usually containing the TMEGF456, was concentrated to 4 ml in Amicon Centricon Plus-20 Filter Unit with Biomax-5 membrane by centrifugation at 3000 rpm (1868 RCF) in a SH-3000 rotor for 30 minutes at 4° C. The retentate was purified by reverse phase chromatography using a Grace Vydac C18 4.6×250 mm 3 μm column (Vydac, Hesperia, Calif.) held at 60° C. The column was equilibrated for at least 12 minutes (4 column volumes) at the starting condition of 90% H₂O/0.1% trifluoroacetic acid (TFA) and 10% acetonitrile (MeCN)/0.1% TFA at a 1 mL/minute flow rate. Four mL samples were injected using a waters 717+Auto-sampler. After the last injection, the solvent was changed from 10% to 26% MeCN/0.1% TFA over 9 minutes. The gradient was then slowed, changing from 26 to 40% of MeCN/0.1% TFA over 21 minutes. TMEGF456 elutes in a broad range between 18-21 minutes as monitored by absorbance at 214 nm. Fractions were collected in 1 mL aliquots using a Gilson FC-204 Fraction Collector and the pH of each fraction was adjusted to 7.0 using 100 μL of 200 mM Tris-HCl, pH 8.0. The samples were then lyophilized in 1100 μL aliquots on a high vacuum line and stored at −80° C. The dry powder was dissolved in a small volume of 1×PBS. Concentration was determined by absorbance at 280 nm and the molar absorption coefficient of 6720 M⁻¹ cm⁻¹ as calculated by the method of Pace et al. (18). Samples were diluted to 10 μg/mL (75 nM) and 500 μL aliquots were frozen until irradiated.

HPLC Separation of Oxidized and Unoxidized Methionine Containing Peptides. Samples were diluted in distilled water to a final concentration of 100 μg/ml before single-dose irradiation (0, 1.77, 10, 20, 40, or 80 Gy). Samples were reduced with an additional 50 mM of fresh TCEP, pH 7.0 from a 1 M stock solution and allowed to mix at room temperature for 5 minutes, and then injected to analyze for oxidation. The HPLC method was optimized to observe 20 μmol of peptide using a Waters Atlantis 4.6×250 mm dC18 5 μm reverse phase column heated to 65° C. The gradient, at a constant 1 mL/minute flow rate, starts at 10% MeCN/0.1% TFA and ramps over eight minutes to 20% MeCN/0.1% TFA then, over an additional 20 minutes, changes to 30% MeCN/0.1% TFA.

Digestion and analysis of rTMEGF456 samples. Each 5 μg TMEGF458 sample was transferred to 1.5 mL micro-centrifuge tube and lyophilized. Samples were then brought up in 40 μL reduction buffer containing 1×PBS, pH 7.4, 50 mM TCEP pH 7.0, and 200 U PNGase F (New England Biolabs, Ipswich. MA). Samples were simultaneously reduced and deglycosotated for 30 minutes at 37° C. Chymotrypsin (Princeton Separations, Adelphia, N.J.) was prepared to manufacturer's specifications at 1 μg/μL in 25 μL of 50 mM Tris-HCl pH 8.0, 1 mM CaCl₂, 2.5 μg chymotrypsin was added to each tube, and the samples were placed in a shaker for 4 hours at 30° C. Alter incubation, the samples were boiled for 45 seconds to deactivate chymotrypsin and stored at −80° C. until analysis by HPLC. Each digest was subjected in triplicate to the HPLC analysis described above.

Mass spectroscopy confirmation. Peak locations and identities in the more complex peptide digest mixture were confirmed using HPLC co-injections of synthetic peptide and by mass spectroscopy. Fractions were taken of peaks thought to correspond to the oxidized and reduced forms. These solutions were mixed with dihydroxybenzoic acid, spotted onto target plates, and allowed to dry before insertion into a Bruker MALDI-TOF Reflex III mass spectrometer (Bruker Daltonics, Billerica, Mass.) for confirmation.

### Statistical Analysis

For each of the parameters (protein C activation, SPR RUs, and Met388 oxidation), the Jonckheere-Terprstra test was used to determine whether there was a radiation dose-dependent increase/decrease, using the CytelStudio/StatXact 8 software package for exact non-parametic inference (Cytel Software, Cambridge, Mass.). The Jonckheere-Terprstra test is similar to the Kruskall-Wallis test (non-parametric one-way analysis of variance), but makes the additional assumption that the populations are not random, but rather exhibit a trend (in this case a radiation dose-dependent trend). Selected univariate comparisons of individual differences were performed with Student's t-test. An alpha level of 0.05 was established as level of significance for all tests.

### Results

### TM Activity

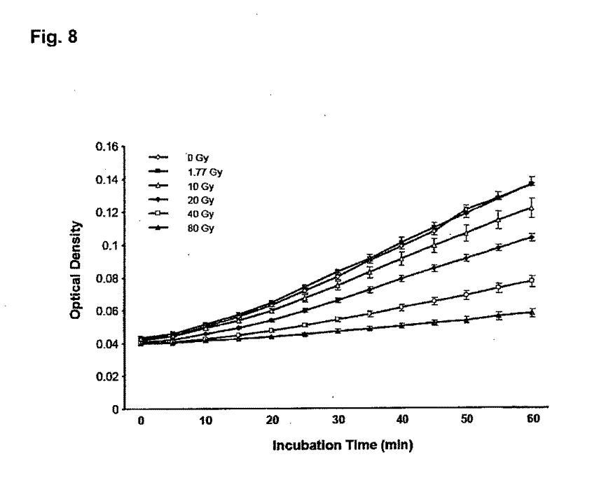

Radiation exposure of recombinant human TM caused a strong, highly statistically significant dose-dependent decrease in TM functional activity as assessed by the protein C activation assay (p=6×10⁻⁸, FIG. 8). There was no decrease in TM activity at 1.77 Gy, whereas, at 10 Gy, the decrease was already significant (p=0.03).

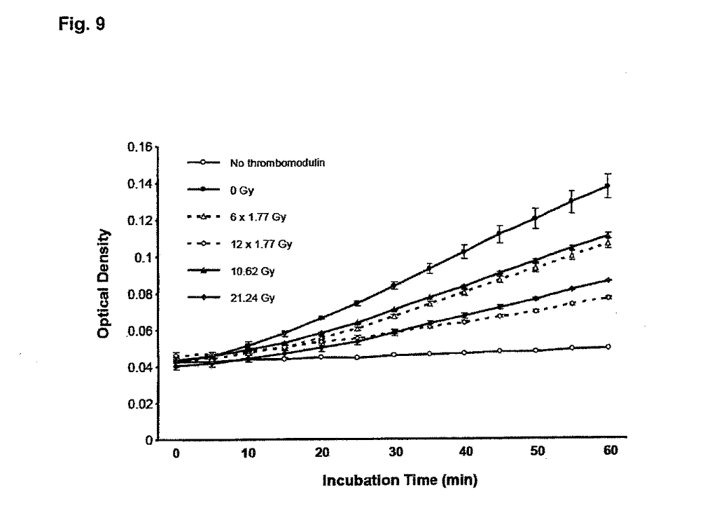

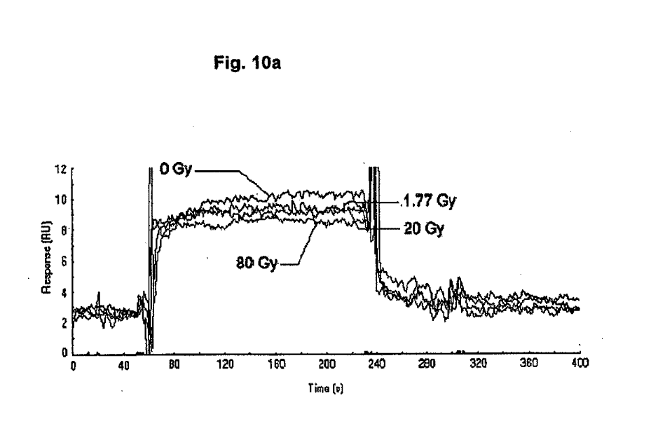

Exposure to fractionated radiation revealed a highly statistically significant reduction in TM's ability to activate protein C, both after 6 fractions (p=0.001) and after 12 fractions (p=0.0001) of 1.77 Gy (FIG. 9). There was no difference in TM activity whether a dose of 10.62 Gy was delivered as a single fraction or as 6 fractions (p=0.11). Interestingly, 21.24 Gy delivered as 12 fractions was associated with significantly greater reduction in protein C activation than when the radiation dose was administered as a single exposure (p=0.003).

MPO enhanced the radiation dose-dependent reduction in protein C activation at every dose level examined (p<0.05 at every dose level). For example, while exposure to 1.77 Gy without MPO caused a mean reduction in protein C activation of 12% in this experiment, the same radiation dose reduced protein C activation by 40% in the presence of MPO.

### Thrombin-TM Complex Formation

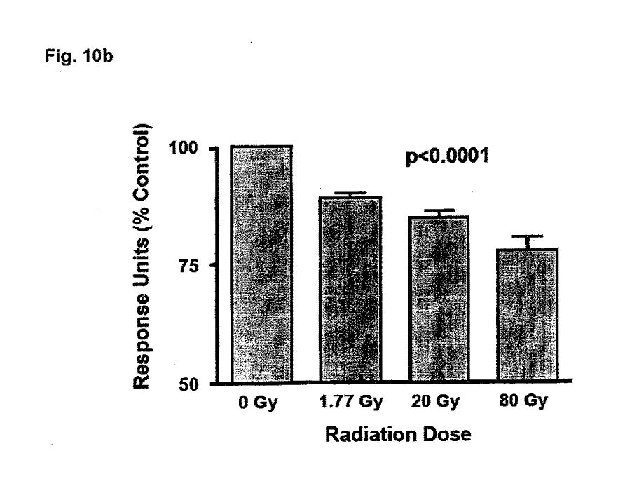

Sensograms (an example is shown in FIG. 10a) were recorded for each experiment. The bar graph depicted in FIG. 10b shows the average RU values after radiation exposure (relative to the 0 Gy sample) from 3 independent experiments. Exposure to ionizing radiation caused a highly statistically significant dose-dependent decrease in the interaction between thrombin and thrombomodulin (p=0.00006). Relative to baseline (0 Gy), the average RU value decreased by 13.2±1.0% after exposure to 1.77 Gy, by 18.9±1.7% after 20 Gy, and by 21.6±1.8% after 80 Gy.

### Oxidation of Met388

Following chymotrypsin digestion of TMEGF456, the resultant peptide was structurally identical to the synthetic peptide. The oxidized peak in both the synthetic peptide and the digested TMEGF456 eluted at 14.7 minutes and the unoxidized peak eluted at approximately 18.3-18.4 minutes. These peaks were baseline separated from the other peaks of the digest.

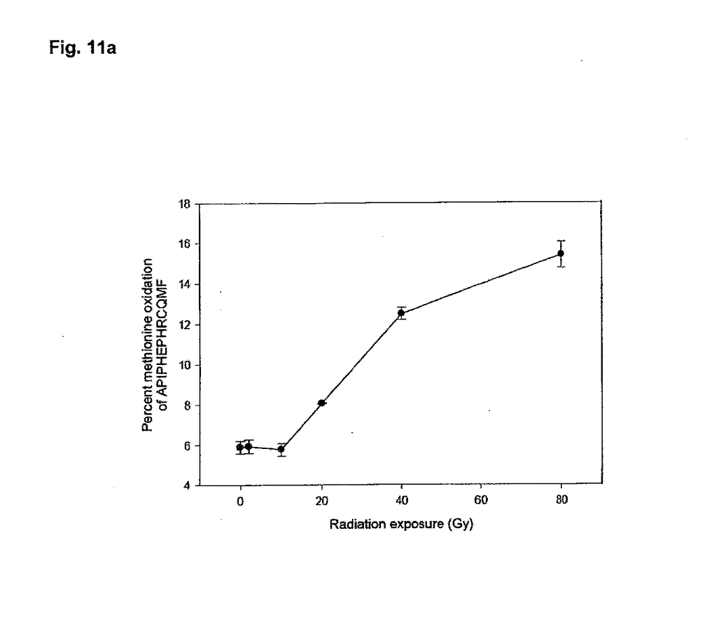

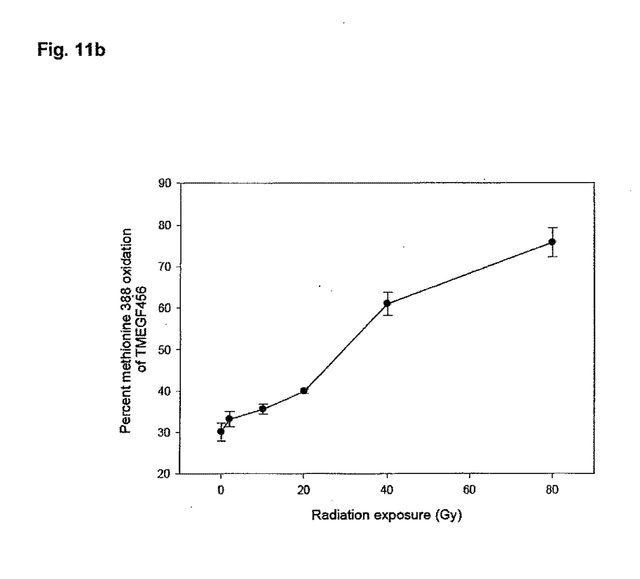

There was a highly statistically significant radiation dose-dependent increase in Met388 oxidation of the synthetic peptide (p=0.0001, FIG. 11a), although there was little change in oxidation at lower levels of radiation exposure.

Samples of the recombinant protein fragment TMEGF456 started out with about 30% of Met388 already in the sulfoxide form. In contrast to the synthetic peptide, TMEGF456 showed increased oxidation even after the lower radiation doses, and exhibited a highly statistically significant increase in Met388 oxidation with increasing radiation exposure (p=4×10⁻⁹, FIG. 11b).

Mass spectroscopy confirmed that the molecular ions were of the expected mass in each case.

### REFERENCE LIST

1. C. B. Glaser, J. Morser, J. H. Clarke, E. Blasko, K. McLean, I. Kuhn, R. J. Chang, J. H. Lin, L. Vilander, et al, Oxidation of a specific methionine in thrombomodulin by activated neutrophil products blocks cofactor activity. J. Clin. Invest. 90, 2565-2573 (1992).
2. A. Kishida, M. Nakashima, N. Sakamoto, T. Serizawa, I. Maruyama, and M. Akashi, Study on the complex formation between recombinant human thrombomodulin fragment and thrombin using surface plasmon resonance. Am J. Hematol. 63, 136-140 (2000).
3. M. J. Wood and E. A. Komives, Production of large quantities of isotopically labeled protein in Pichia pastoris by fermentation. J. Biomol. NMR 13, 149-159 (1999).

### LEGEND

FIG. 1.: Whole body irradiation of mice. Veh., vehicle controls; Sol., solulin treatment

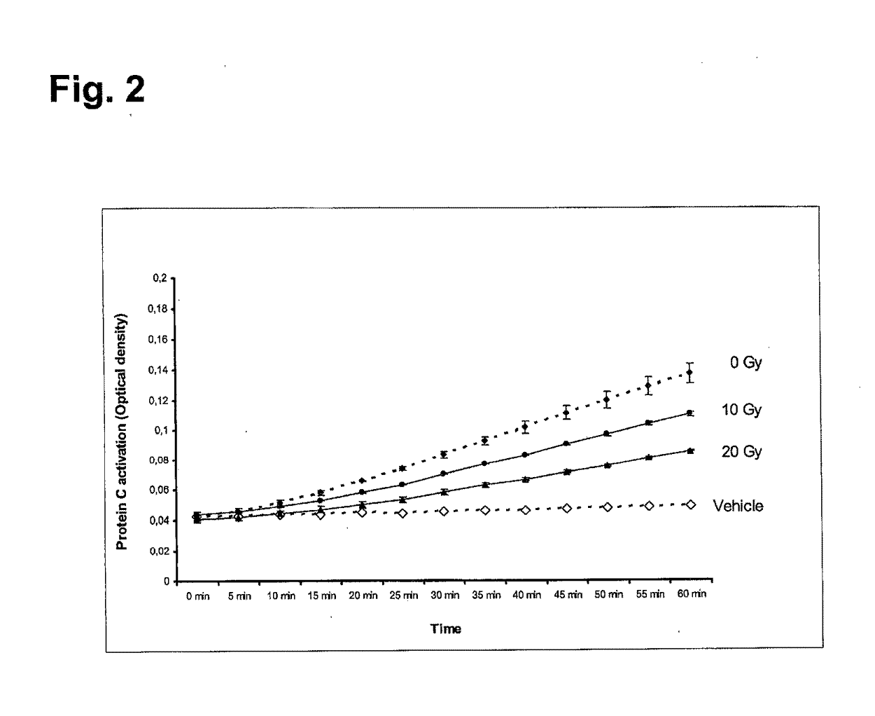

FIG. 2.: Irradiation of recombinant human TM (2.5 nM), single dose

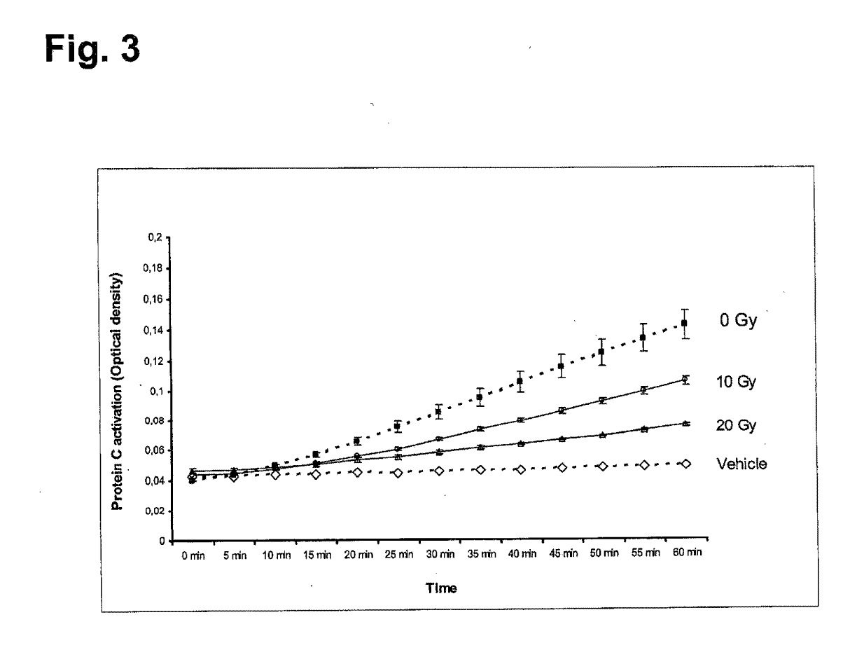

FIG. 3.: Irradiation of recombinant human TM (2.5 nM), fractionated dose

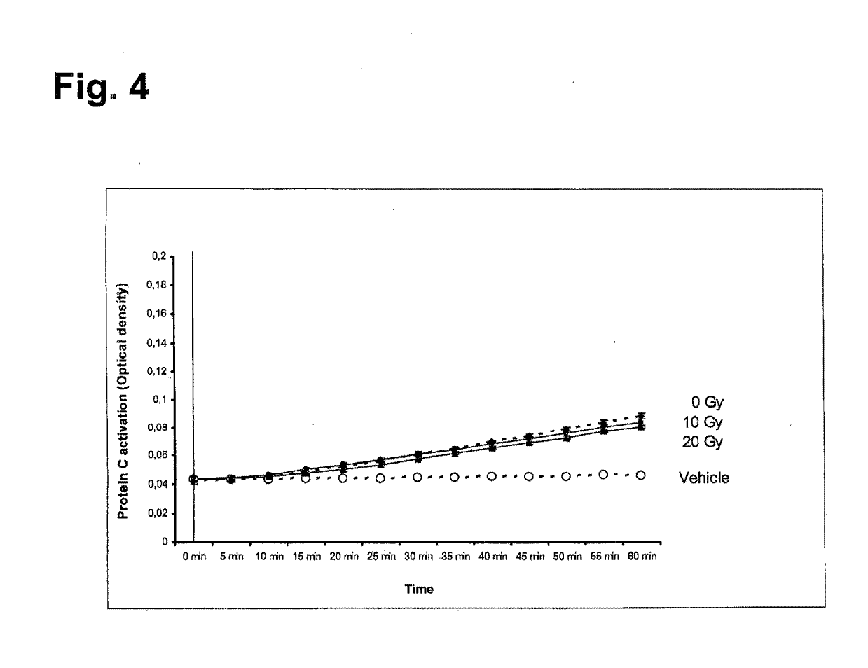

FIG. 4.: Irradiation of solulin (2.5 nM), single dose

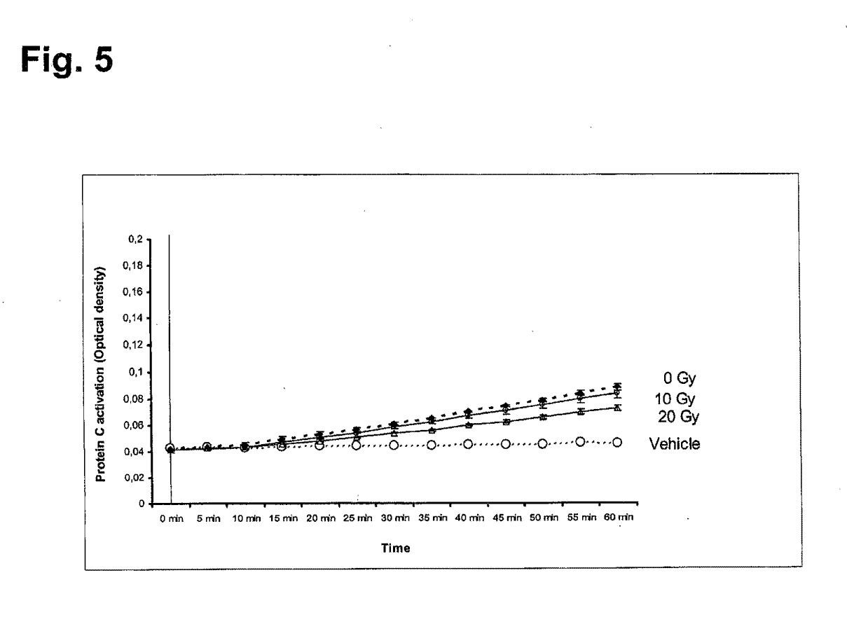

FIG. 5.: Irradiation of solulin (2.5 nM), fractionated dose

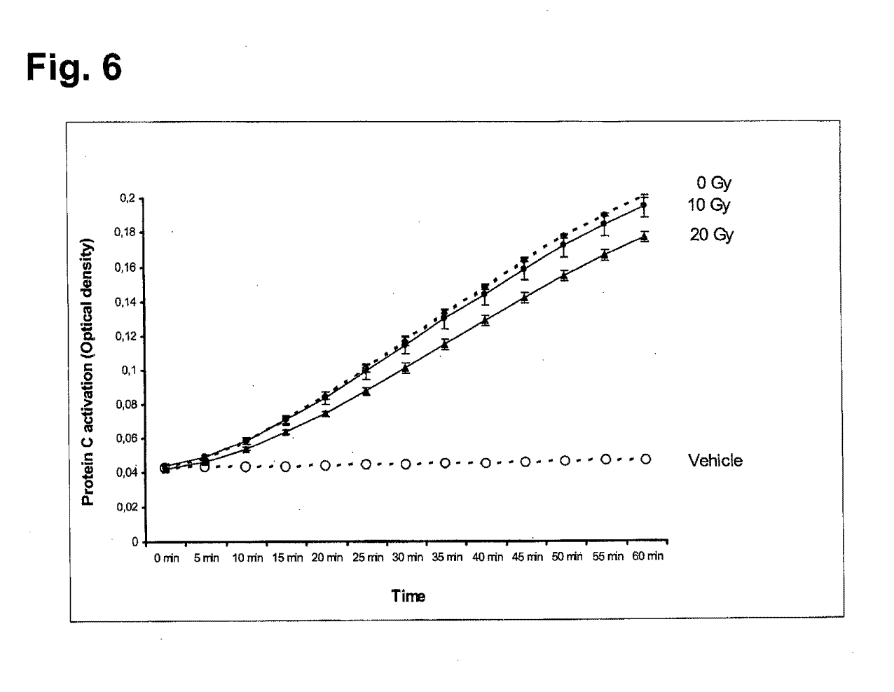

FIG. 6.: Irradiation of solulin (5.0 nM), single dose

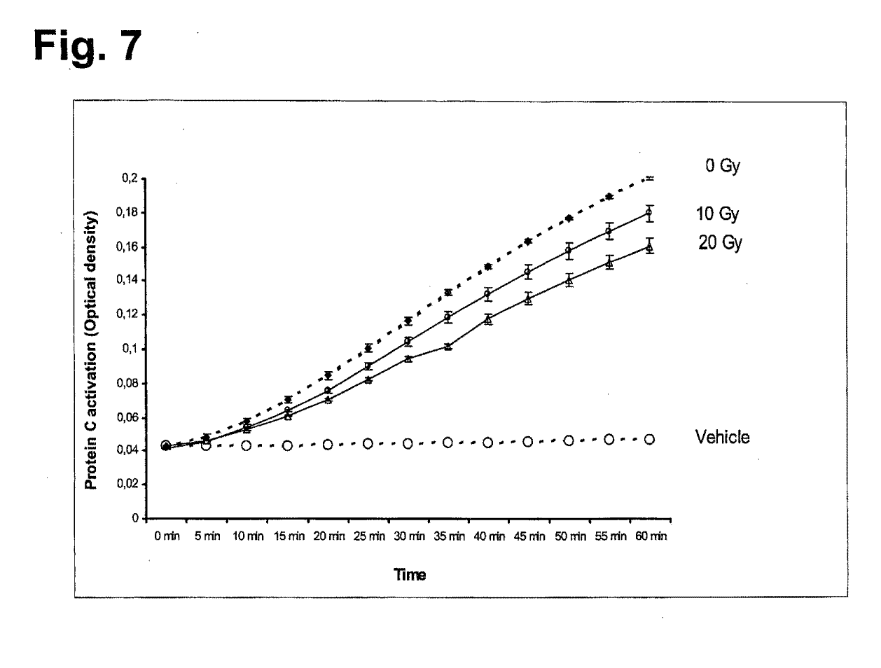

FIG. 7.: Irradiation of solulin (5.0 nM), fractionated dose

FIG. 8.:

Functional Activity of TM Exposed to Single Dose Irradiation.

There is a highly statistically significant radiation dose-dependent inhibition of TM's cofactor function as measured by the protein C activation assay (p=6×10⁻⁸). Data points represent mean and standard error of 3 independent samples.

FIG. 9.:

Functional Activity of TM Exposed to Fractionated Irradiation.
TM's ability to activate protein C is highly significantly reduced, both after 6 fractions of 1.77 Gy (p=0.001), as well as after 12 fractions of 1.77 Gy (p=0.0001). There is no difference in TM activity whether the dose of 10.62 Gy was delivered as a single fraction or as 6 fractions (p=0.11), whereas, 21.24 Gy delivered as 12 fractions was associated with significantly greater reduction in protein C activation than when administered as a single dose (p=0.003). Data points represent mean and standard error of 3 independent samples.

FIGS. 10a and 10b:

Surface Plasmon Resonance (SPR) analysis
Panel A: Overlay of 4 different sensograms to demonstrate how the SPR response of TM exposed to different doses of radiation can be compared.
Panel B: Average TM-thrombin complex formation as a function of radiation dose (mean and standard error for response units [RU] obtained from sensograms, as percent of sham-irradiated control). There is a highly statistically significant radiation dose-dependent decrease in the association between TM and thrombin (p=0.00006). Average and standard error of 3 separate experiments.

FIGS. 11a and 11b:

Oxidation of Methionine 388 (Met388) as a Function of Radiation Dose.
Panel A: Met388 oxidation in synthetic peptide as a function of radiation dose.
Panel B: Met388 oxidation in the recombinant TME456 fragment as a function of radiation dose. Data points are mean and standard deviation from at least 3 separate samples. There are highly statistically significant radiation dose-dependent increases in Met388 oxidation both of the synthetic peptide and of TME456 (p=0.0001 and p=4×10⁻⁹, respectively).

1. Use of thrombomodulin or a thrombomodulin analogue for the manufacture of a medicament for the treatment of radiation injury.

2. Use of thrombomodulin or a thrombomodulin analogue for the manufacture of a medicament for the prevention of radiation injury.

3. Use of thrombomodulin or a thrombomodulin analogue for the manufacture of a medicament for the treatment of patients subject to radiotherapy for cancer.

4. Use of thrombomodulin or a thrombomodulin analogue for the manufacture of a medicament for the treatment of cancer survivors.

5. Pharmaceutical composition comprising a therapeutically effective amount of component A selected from thrombomodulin or a thrombomodulin analogue and a component B selected from a group of cytotoxic agents exhibiting oxidizing potential.

6. Use according to claim 1, whereas the thrombomodulin analogue has an amino acid sequence corresponding to the amino acid sequence of mature soluble thrombomodulin SEQ ID No1 and comprises one or more of the subsequent modifications:
removal of amino acids 1-3
M388L.
R456G
H457Q
S474A, and terminating at P490.

7. Use of the pharmaceutical composition of claim 5, whereas the thrombomodulin analogue has an amino acid sequence corresponding to the amino acid sequence of mature soluble thrombomodulin SEQ ID No1 and comprises one or more of the subsequent modifications:
removal of amino acids 1-3
M388L
R456G
H457Q
S474A, and terminating at P490.


SEQUENCE LISTING (2 sequences)

SEQ ID NO: 1 - protein, 575 aa, Homo sapiens
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALVRHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 2 - DNA, 1518 bp, Homo sapiens
atgcttggggtcctggtccttggcgcgctggccctggcaggcctggtcttccccgagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagctgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgctggccagattggcaccgactgtgactccggcaaggtggacggtggcgacagcggcgccggcgagcccccgcccagcccgacgcccgggtctaccttgactcctccgtga